(19) United States
(12) Patent Application Publication
(10) Pub. No.: US 2005/0048122 A1
(43) Pub. Date: Mar. 3, 2005

(54) Method for stabalizing timolol concentration

(75) Inventors: Paul Philip Grabowski, Portage, MI (US); Wayne Thomas Leblong, Portage, MI (US); Robert Forrest Landeryou, Kalamazoo, MI (US)

Correspondence Address: AGOURON PHARMACEUTICALS, INC., 10350 NORTH TORREY PINES ROAD, LA JOLLA, CA, 92037, US

(73) Assignee: Pharmacia & Upjohn Company

(21) Appl. No.: 10/871,770

(22) Filed: Jun. 18, 2004

Related U.S. Application Data
(60) Provisional application No. 60/479,683, filed on Jun. 19, 2003.

(51) Int. Cl. A61K 9/14; A61K 31/5377

(52) U.S. Cl. 424/486; 514/235.5

(57) ABSTRACT

A novel method for stabilizing timolol concentration in a container containing an aqueous composition comprising timolol is disclosed. The container is made of a pharmaceutically acceptable, moisture-permeable material. This method comprises the step of enclosing the container in a secondary package. The secondary package is made of a pharmaceutically acceptable material having a low permeability to moisture. A medicinal product comprising an aqueous composition comprising timolol is also disclosed. The composition is packaged in a container made of a pharmaceutically acceptable, moisture-permeable material. The container is enclosed in a secondary package, which is made of a pharmaceutically acceptable material having a low permeability to moisture.

### TECHNICAL FIELD

This application claims benefit of U.S. Provisional Patent Application No. 60/479,683 filed Jun. 19, 2003. The present invention concerns the field of packages for medicinal products. More specifically, the present invention relates to an improved package for a container containing an aqueous composition comprising timolol. The present invention is also concerned with a method of maintaining the concentration of one or more of the active components in the aqueous composition.

### BACKGROUND

Medicinal products must possess certain levels of stability and purity in order to be suitable for safe and efficacious administration to patients. Medicinal products are considered stable if the concentration of active ingredient(s) can be maintained at the level specified on the label for the maximum anticipated shelf-life under given environmental conditions. A medicinal product is considered unstable when the active ingredient or excipients, such as preservatives, flavoring agents, etc, loses sufficient potency to adversely affect the safety or efficacy of the drug or falls outside labeled specifications.

The potency of a drug product may decline over time during storage due to various reasons, such as degradation of the active ingredient(s), reaction of the active ingredient(s) with excipients or container materials, leaching of the active ingredient(s) through the container wall, and absorption of the active ingredient(s) into the container wall. Similarly, the purity of a medicinal preparation may also change during storage due to leaching of chemicals into the drug preparation from the container materials, from the labels on the containers, or from the environment where the packaged medicinal product is stored. Thus, the containers used for packaging medicinal preparations can significantly affect the stability and purity of the preparations contained therein.

Containers commonly used for medicinal products include glass containers, polypropylene containers, and polyethylene containers. (See e.g. U.S. Pat. No. 6,235,781). However, since glass containers are rigid and not squeezable, they are not very suitable for medicinal preparations, which are conveniently dispensed on a drop-by-drop basis. Typical user-friendly containers, dispensers, or bottles for medicinal preparations are formed from e.g. polyethylene, polypropylene, or polyethylene terephthalates (PET), which in most instances provide a suitable combination with a pharmaceutical preparation, resulting in a packaged medicinal product that is user-friendly for dispensing of the pharmaceutical preparation on a drop-by-drop basis.

The ophthalmic products XALATAN® (comprising approximately 50 μg/ml latanoprost) and XALCOM® (comprising approximately 50 μg/ml latanoprost and 5 mg/ml timolol) are packaged in bottles. The bottle is typically made of clear low-density polyethylene with a clear low-density polyethylene dropper tip, a high-density polyethylene screw cap, and a clear low-density polyethylene overcap. Unopened bottles are stored at 2-8° C. Upon storage at the recommended temperature, the concentrations of latanoprost and latanoprostltimolol, respectively, are stably maintained in the products.

While storage at recommended temperature has proven to be an effective way of maintaining product stability of the ophthalmic products, it would be desirable to store such ophthalmic products at room temperature for prolonged periods without adversely affecting the concentration(s) of the active ingredient(s). In particular, it has been observed that the latanoprost concentration is maintained at a nearly constant level in the current polyethylene bottles, both at low (2-8° C.) and at room temperature. Curiously, the timolol concentration is maintained at a nearly constant level in the current polyethylene bottles at low (2-8° C.) temperature, but it increases gradually over time at room temperature.

### SUMMARY OF THE INVENTION

The present invention provides a method for stabilizing timolol concentration in a container containing an aqueous composition comprising timolol, which container is made of a pharmaceutically acceptable, moisture-permeable material, said method comprising the step of enclosing said container in a secondary package, which is made of a pharmaceutically acceptable material having a low permeability to moisture.

According to another aspect, the present invention provides a medicinal product comprising an aqueous composition comprising timolol packaged in a container, which container is made of a pharmaceutically acceptable, moisture-permeable material, wherein said container is enclosed in a secondary package, which is made of a pharmaceutically acceptable material having a low permeability to moisture.

In one embodiment of the method or medicinal product according to the invention, said aqueous composition is an ophthalmic composition.

In an embodiment of the method or medicinal product according to the invention, said aqueous composition further comprises a prostaglandin, or an analogue or derivative thereof. In a preferred embodiment of the method or medicinal product according to the invention, said aqueous composition comprises latanoprost.

In one embodiment of the method or medicinal product according to the invention, said container is at least partially made of a plastic material selected from the group consisting of polymers and copolymers of polyvinyl chloride, polyethylene terephthalates (PET), PET copolyester (PETG), polyolefins, and combinations thereof.

In a specific embodiment of the method or medicinal product according to the invention, said plastic material is polyethylene or polypropylene.

In an embodiment of the method or medicinal product according to the invention, said secondary package is made of a material selected from the group consisting of homopolymers or copolymers of chiorotrifluoroethylene (CTFE), polyvinylidene chloride (PVDC), polyethylene vinyl alcohol, polyolefins, polyvinyl chloride, polyethylene terephthalates (PET), PET copolyester (PETG), and combinations thereof.

In a specific embodiment of the method or medicinal product according to the invention, said secondary package is made of a material comprising polyvinyl chloride in combination with homopolymers or copolymers of CTFE or PVDC. Optionally, said secondary package material comprises polyolefins in combination with polyethylene vinyl alcohol or PVDC.

In one embodiment of the method or medicinal product according to the invention, said secondary package is a sealed blister package.

As used herein, the term “medicinal preparation” refers to matter of compositions, whose biological, physiological, pharmacological, or chemical activities are beneficial for animals or humans in normal or pathological conditions, such as diagnosis, prognosis, treatment, prophylaxis, therapy, or for animal production.

Timolol is a non-selective beta-adrenergic receptor blocking agent. The chemical name for timolol maleate is (S)-1-[(1,1-dimethylethyl)amino]-3-[ [4-(4-morpholinyl)-1,2,5-thiadiazol-3-yl]oxy]-2-propanol, or (S)-1-tert-butylamino-3-(4-morpholino-1,2,5-thiadiazol-3-yloxy) propan-2-ol hydrogen maleate. Timolol decreases intra-ocular pressure and is useful for treatment of certain ophthalmic conditions, such as glaucoma.

As used herein, the term “prostaglandin” or “PG” shall refer to prostaglandins and derivatives and analogues thereof, including pharmaceutically acceptable salts and esters, except as otherwise indicated by context.

By the expression “stabilizing timolol concentration” is herein meant that the timolol concentration in the composition is maintained at the desired concentration in the container upon storage at room temperature and that a gradual increase in timolol concentration is prevented. The timolol concentration will he maintained at a level that is less than 10% (w/v), preferably less than 5% more preferably less than 2%, from the desired concentration upon storage at room temperature for a period of from one to five years, such as about two years. The desired timolol concentration may be in the range of 0.1-100 mg/ml, preferably 1-20 mg/m1.

In one preferred embodiment of the invention, the secondary package is made of a material comprising homopolymers or copolymers or chlorotrifluoroethylene (CTFE). Optionally, the secondary package material further comprises polyvinylchloride.

The secondary package material may comprise of a combination of polyvinyl chloride and polychiorotrifluoroethylene, which is known by the trade name ACLAR® or TEKLAR@. The latter polymer provides a high moisture barrier.

As depicted in FIG. 1, the secondary package is a sealed blister package (2). The container (1), such as a bottle, is enclosed in the blister (2). The blister (2) is formed to hold the container (1). The blister (2) is sealed, optionally with a metal foil, e.g. aluminum foil or aluminum foil lined board. The sealed blister (2) provides a moisture barrier for the container (1) comprising the medicinal composition according to the invention.

The secondary package is made of a substantially clear material. The secondary package can be made by a thermoforming process, injection molding, or compression molding.

The method disclosed herein is particularly useful when the aqueous composition is an ophthalmic composition, since the inventive method allows for the use of readily squeezable container materials. Such containers are particularly useful for treatment of ophthalmic conditions, including glaucoma.

The aqueous composition described herein comprises timolol and a prostaglandin, or an analogue or derivative thereof. As used herein, the term “prostaglandin” or “PG” shall refer to prostaglandins and derivatives and analogues thereof, including pharmaceutically acceptable salts and esters, except as otherwise indicated by context. Prostaglandins may be classified according to their 5-membered ring structure, using a letter designation. Prostaglandins may be further classified based on the number of unsaturated bonds in the side chain. The prostaglandins that may be utilized in the present invention include all pharmaceutically acceptable prostaglandins, their derivatives and analogues, and their pharmaceutically acceptable esters and salts. Such prostaglandins include the natural compounds PGE₁, PGE₂, PGE₃, PGF₁ₐ, PGF₂ₐ, PGF₃ₐ, PGD₂, and PGI₂ (prostacyclin), as well as analogues and derivatives of these compounds which have similar biological activities.

Analogues of the natural prostaglandins include, but are not limited to, compounds resulting from modifications of the omega chain (e.g. 18,19,20-trinor-17-phenyl, 17,18,19,20-tetranor-16-phenoxy), which enhance selectivity of action and reduce ocular side-effects.

Especially preferred prostaglandin derivatives are physiologically acceptable derivatives of prostaglandin PGF, in which the omega chain has the formula: 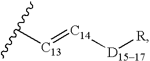
wherein

C is a carbon atom (the carbon atom number is indicated as a subscript); the bond between C₁₃ and C₁₄ is a single bond or a double bond;

D is a chain with 2-3 carbon atoms, optionally interrupted by hetero atoms O, S, or N, the substituents on each carbon atom selected from H, lower alkyl groups with 1-5 carbon atoms, halogens, keto or a hydroxyl group; and

R is (i) a phenyl group which is unsubstituted or has at least one substituent selected from C₁-C₅ alkyl groups, C₁-C₄ alkoxy groups, trifluoromethyl groups, C₁-C₃ aliphatic acylamino groups, nitro groups, halogen atoms, or a phenyl group; or
(ii) an aromatic heterocyclic group having 5-6 ring atoms, such as thiazol, imidazole, pyrrolidine, thiphene and oxazole which is unsubstituted or has at least one substituent selected from C₁-C₅ alkyl groups, C₁-C₁ alkoxy groups, trifluoromethyl groups, C₁-C₃ aliphatic acylamino groups, nitro groups, halogen atoms, or a phenyl group; or
(iii) a cycloalkyl or a cycloalkylene group with 3-7 carbon atoms, optionally substituted with 1-5 carbon atoms.

Most preferably, the prostaglandin derivatives are 17-phenyl-18,19,20-trinor derivatives, 13,14-dihydro- 17-phenyl-18,19,20-trinor derivatives, 16-phenoxy-17,18,19,20-tetranor derivatives, or 16-phenyl-17,18,19,20-tetranor derivatives.

Derivatives of these prostaglandins include all pharmaceutically acceptable salts, esters (alkyl esters, such as isopropyl esters) and amides (lower alkyl amides, such as N-diethyl amide) that may be attached to the 1-carhoxyl group or any of the hydroxyl groups of the prostaglandin by use of the corresponding alcohol or organic acid reagent, as appropriate. It should be understood that the terms “analogues” and “derivatives” include compounds that exhibit functional and physical responses similar to those of prostaglandins per se.

Specific examples of prostaglandins that are useful in the present invention include the following compounds:
1. 17-phenyl-18,19,20-trinor-PGF₂ₐ-isopropyl ester;
2. 15-(R)-17-phenyl-18,19,20-trinor-PGF₂ₐ-isopropyl ester;
3. 16-phenyl-17,18,1 9,20-tetranor-PGF₂ₐ-isopropyl ester;
4. 20-pentanor-13-prostynoic acid;
5. latanoprost;
6. cloprostenol isopropyl ester;
7. (5Z)-(9S, 11R, 15S)-15-cyclohexyl-9,11,15-trihydroxy-16,17,18,19,20-pentanor-5-prostenoic acid isopropyl ester;
8. (5Z, 13E)-(9S, 11R, 15R)-9,11,15-trihydroxy-16-(3-chlorophenoxy)-17,18,19,20-tetranor-5,13-prostadienoic acid amide;
9. PGF₂ₐ isopropyl ester;
10. fluprostenol isopropyl ester;
11. isopropyl [2R(IE,3R),3S(4Z),4R1-7-[tetrahydro-2-[4-(3-chlorophenoxy)-3-hydroxy-1-butenyl]-4-hydroxy-3-furanyl]-4-heptenoate;
12. 15-keto latanoprost;
13. bimatoprost;
14. unoprostone isopropyl;
15. 15-deoxy-15-fluro-16-phenoxy-17,18,19,20-tetranor-PGF₂ₐ isopropyl ester;
16. 15-deoxy- 15-fluro-16-(3-chlorophenoxy)-17,18,19 ,20-tetranor-PGF₂ₐ, isopropyl ester;
17. 15-deoxy-15-fluro-16-(3-trifluoromethylphenoxy)-17,18,19,20-tetranor-PGF₂ₐ isopropyl ester;
18. 15-deoxy- 15-difluro-16-phenoxy-17,18,19,20-tetranor-PGF₂ₐ-isopropyl ester;
19. 15-deoxy-15-difluro-16-(3-chlorophenoxy)-17,18,19,20-tetranor-PGF₂ₐ isopropyl ester;
20. 15-deoxy-15-difluro-16-(3-trifluoromethylphenoxy)-17,18,19,20-tetranor-PGF₂ₐ isopropyl ester;
21. 15-deoxy-15-difluro-16-phenoxy-17,18,19,20-tetranor-PGF₂ₐ, isopropyl ester; and
22. DE-085.

### BRIEF DESCRIPTION OF THE DRAWINGS

FIG. 1 shows a medicinal product according to the invention, consisting of a bottle-type container, comprising an aqueous composition, enclosed in a blister package.

### DETAILED DESCRIPTION OF THE INVENTION

Upon storage of compositions comprising prostaglandin and prostaglandin/timolol, respectively, it has been observed that the prostaglandin concentration is maintained at a nearly constant level in polyethylene bottles, both at low (2-8° C.) and at room temperature. It has also been observed that the timolol concentration is maintained at a nearly constant level in polyethylene bottles at low (2-8° C.) temperature, but increases gradually over time at room temperature.

While storage at low temperature has proven to be an effective way of maintaining product stability of the known medicinal products, it would be desirable to store such medicinal products at room temperature for prolonged periods of time without adversely affecting the concentration(s) of the active ingredient(s). Thus, the problem that has been identified is that the relative concentrations of prostaglandin and timolol differ over time upon storage at room temperature. In particular, the concentration of timolol increases over time upon storage at room temperature. This may cause higher dosing than desired, leading to common overdosage effects associated with systemic beta-adrenergic receptor blocking agents, e.g. dizziness, headache, shortness of breath, bradycardia, and bronchospasm.

Following extensive research, a better understanding of the above-mentioned phenomenon has now been achieved. When the polyethylene packages are stored, moisture from the composition is lost to the atmosphere. Moreover, prostaglandin itself is absorbed into or adsorbed to the bottle resin and thus removed from the composition. Surprisingly, the result of these two separate events is a stabilization of the overall concentration of prostaglandin over time. In contrast, timolol is neither lost to the atmosphere nor absorbed/adsorbed by the package material to any large extent. Thus, the concentration of timolol increases over time and may have undesired systemic effects upon administration to the eye. This net effect of this phenomenon is more pronounced at room temperature than at low temperature.

According to the invention, the increase in timolol concentration in a container containing an aqueous composition comprising timolol can be reduced by arranging said container in a secondary package that has a high barrier to moisture. This secondary package will thus provide a moisture barrier that prevents a gradual increase in the concentration of timolol over time.

The container comprising the aqueous composition of timolol is made of a pharmaceutically acceptable, moisture-permeable material.

The term “pharmaceutically acceptable” as used herein refers to materials that are acceptable for containment of pharmaceutical substances by virtue of the relative inertness of the material, meeting specific requirements established by the FDA.

The term “moisture-permeable” as used herein refers to materials that are permeable to moisture, in particular to water vapor. Examples of such materials include, but are not limited to, polymers and copolymers of polyvinyl chloride, polyethylene terephthalates (PET), PET copolyester (PETG). and polyolefins. Polyolefins include e.g. polyethylene, polypropylene, polybutenes, polyisoprenes and polypentenes, and copolymers and combinations thereof.

An example of a suitable material is polyethylene or a blend of polyethylene and one or more other materials. Polyethylene is commonly divided into classes based on its density. Classes commonly used include low-density polyethylene (LDPE), medium-density polyethylene (MDPE) and high-density polyethylene (HDPE). This list of classifications should not be considered as a standard or a complete list of classifications. Given these rather loose classifications, polymer characteristics vary among multiple producers of a given class of polyethylene, or among multiple grades of a given class by one producer.

The container can be made of polyethylene of any density, a blend of polyethylenes of various densities, or a blend of polyethylene with other materials. For medicinal preparations that are desirably packaged in squeezable containers, particularly ophthalmic medications, it is advantageous that the container of the invention is made of a material comprising LDPE. Depending on the desired level of squeezability of the container, the relative content of LDPE in the container materials can be adjusted accordingly. Generally, containers made of LDPE are more readily squeezable than container made of MDPE or HDPE. Similarly, containers made of materials having a relatively high content of LDPE are more readily squeezable than containers made of materials having a relatively low content of LDPE.

Alternatively, the container according to the invention can be made of polypropylene or a blend of polypropylene with other materials. The term polypropylene includes e.g. isotactic polypropylene, syndiotactic polypropylene, and blends of isotactic and syndiotactic polypropylene. The use of polypropylene is particularly advantageous when the composition encompasses a prostaglandin, or an analogue or derivative thereof.

The shape, style, and/or size of containers for use with the present invention are not important. For example, the container can be a bottle, a vial, or a syringe. According to a preferred embodiment, the container is preferably a “small volume” bottle. As used herein, the term “small volume” bottle shall mean a bottle of a size sufficient to hold a quantity of liquid medicine sufficient for 1-3 topical doses per day over 1-2 months, generally about 20 ml or less. By way of example, small volume containers include 2.5 ml, 5 ml, 10 ml and 15 ml sized bottles, designed for topically administering eye drops. Small volume bottles made of LDPE are easier to squeeze than larger bottles, and oval bottles are easier to squeeze than round bottles. Accordingly, liquid preparations adapted for topical ophthalmic administration are preferably packaged in oval LDPE bottles.

According to the invention, the container is enclosed in a secondary package, which is made of a pharmaceutically acceptable material having a low permeability to moisture. In other words, the container constitutes an inner vessel, which is encompassed by an outer vessel, i.e. the secondary package. Suitably, the secondary package is made of a thermoforinable plastic material.

The term “low permeability to moisture” as used herein refers to materials that are less permeable to moisture than high-density polyethylene (HDPE). Preferably, the employed materials are less permeable to moisture than polyvinyl chloride. Specifically, the secondary package according to the invention, which is made of a pharmaceutically acceptable material having a low permeability to moisture, is substantially less permeable to moisture than the container according to the invention, which is made of a pharmaceutically acceptable, moisture-permeable material. In particular, low permeability to moisture includes low permeability to water vapor, e.g. a secondary package material with a water vapor transmission rate (WVTR), which is from twofold to tenfold lower than the primary package material WVTR.

The WVTR values for some typical polymers are as follows. PP has a WVTR=0.2 g/m²/24 h at 20° C. and 85% relative humidity (RH); PET has a WVTR=2.6 g/m²/24 h at 20° C. and 85% RH; and PVC has a WVTR=1.1 g/m²/24 h at 20° C. and 85% RH. If the storage temperature and relative humidity are increased, the moisture transmission rates change. For example, monolayer PVC has a WVTR=3.10--3.78 g/m²24 h at 38° C. and 90% RH. Higher moisture barrier is attained by the combination of PVC with polyvinylidene chloride (PVDC) or chlorotrilluoroethylene (CTFE). PVC/PVDC (40 g) has a WVTR=0.60 g/m²24 h at 38° C. and 90% RH, and PVC/PVDC (60 g) has a WVTR=0.40 g/m²/24 h at 38° C. and 90% RH. PVC/CTFE (250 μ75 μ) has a WVTR=0.07 g/m²/24 h at 38° C. and 90% RH.

Examples of pharmaceutically acceptable materials having a low permeability to moisture include polyvinyl chloride in combination with homopolymers or copolymers of PVDC and/or CTFE. Other examples of pharmaceutically acceptable materials having a low permeability to moisture include polyolefins in combination with polyethylene vinyl alcohol or with PVDC.

All of the foregoing compounds are known. In a preferred embodiment of the invention, the aqueous composition comprises a combination of timolol and latanoprost. The latanoprost concentration may he in the range of 1-1000 μg/ml, preferably 10-100 μg/ml and the timolol concentration may he in the range of 0.1-100 mg/ml, preferably 1-20 mg/ml.

Without being limited thereto, the present invention will in the following be further illustrated by way of examples.

### EXAMPLES

### Example 1

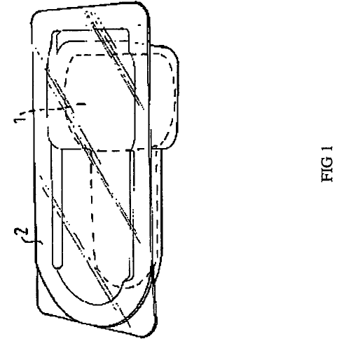

With reference to FIG. 1, a form/fill/seal polyethylene bottle (1), filled with XALCOM® (50 μg/ml latanoprost, timolol maleate corresponding to 5 mg/ml timolol, 0.2 mg/ml benzalkonium chloride (preservative), and sodium chloride in a phosphate buffered solution), is enclosed in a blister (2).

The blister (2) is a clear material, ACLAR® (PVC/CTFE), which is shaped to hold the bottle (1). The blister (2) is sealed with an aluminum foil or aluminum foil lined board (not shown in FIG. 1). The foil side is coated with adhesive to adhere to the blister. The board is sealed to the blister and folded around the blister to box in the blister and allow the package to stand. The sealed blister provides a moisture barrier for the XALCOM® product.

### Example 2

A polypropylene vessel, filled with XALCOM® (50 μg/ml latanoprost, timolol maleate corresponding to 5 mg/ml timolol, 0.2 mg/ml benzalkonium chloride (preservative), and sodium chloride in a phosphate buffered solution), is enclosed in a sealed blister made of ACLAR® (PVC/CTFE). The sealed blister provides a moisture barrier for the XALCOM® product.

### Example 3

Bottles made of polyethylene and bottles made of polypropylene are filled with compositions of timolol in combination with commercially available prostaglandin analogues travoprost, bimatoprost, and unoprostone isopropyl, Each bottle, comprising timolol in combination with one of the analogues, is enclosed in a sealed blister made of ACLAR® (PVC/CTFE).

### Example 4

A form/fill/seal polyethylene bottle, filled with XALCOM® (50 μg/ml latanoprost, timolol maleate corresponding to 5 mg/ml timolol, 0.2 mg/ml benzalkonium chloride (preservative), and sodium chloride in a phosphate buffered solution), is enclosed in a blister.

The blister is a clear material, PVC/PVDC, which is shaped to hold the bottle. The blister is sealed with an aluminum foil or aluminum foil lined board. The foil side is coated with adhesive to adhere to the blister. The board is sealed to the blister and folded around the blister to box in the blister and allow the package to stand. The sealed blister provides a moisture barrier for the XALCOM® product.

### Example 5

A form/fill/seal polyethylene bottle, filled with XALCOM® (50 μg/ml latanoprost, timolol maleate corresponding to 5 mg/ml timolol, 0.2 mg/ml benzalkonium chloride (preservative), and sodium chloride in a phosphate buffered solution), is enclosed in a blister.

The blister is a clear material, polyolefin with PVDC, which is shaped to hold the bottle. The blister is sealed with an aluminum foil or aluminum foil lined board. The foil side is coated with adhesive to adhere to the blister. The board is sealed to the blister and folded around the blister to box in the blister and allow the package to stand. The sealed blister provides a moisture harrier for the XALCOM® product.

### Example 6

A form/fill/seal polyethylene bottle, filled with XALCOM® (50 μg/ml latanoprost, timolol maleate corresponding to 5 mg/ml timolol, 0.2 mg/ml benzalkonium chloride (preservative), and sodium chloride in a phosphate buffered solution), is enclosed in a blister.

The blister is a clear material, polyolefin with ethylene vinyl alcohol (EVOH), which is shaped to hold the bottle. The blister is sealed with an aluminum foil or aluminum foil lined hoard. The foil side is coated with adhesive to adhere to the blister. The board is sealed to the blister and folded around the blister to box in the blister and allow the package to stand. The sealed blister provides a moisture barrier for the XALCOM® product.

### Example 7

A form/fill/seal polyethylene bottle, filled with XALCOM® (50 μg/ml latanoprost, timolol maleate corresponding to 5 mg/ml timolol, 0.2 mg/ml benzalkonium chloride (preservative), and sodium chloride in a phosphate buffered solution), is enclosed in a blister.

The blister is a clear material, polyolefin with ethylene vinyl alcohol (EVOH), which is shaped to hold the bottle. The blister is sealed with paper backed aluminum foil with heat seal coating. The foil side is coated with adhesive to adhere to the blister. The board is sealed to the blister and folded around the blister to box in the blister and allow the package to stand. The sealed blister provides a moisture barrier for the XALCOM® product.

1. A method for stabilizing timolol concentration in a container containing an aqueous composition comprising timolol, which container is made of a pharmaceutically acceptable, moisture-permeable material, said method comprising the step of enclosing said container in a secondary package, which is made of a pharmaceutically acceptable material having a low permeability to moisture.

2. A method according to claim 1, wherein said aqueous composition is an ophthalmic composition.

3. A method according to claim 1, wherein said aqueous composition further comprises a prostaglandin or an analogue or derivative thereof.

4. A method according to claim 1, wherein said aqueous composition further comprises latanoprost.

5. A method according to claim 1, wherein said container at least partially is made of a plastic material selected from the group consisting of polymers and copolymers of polyvinyl chloride, polyethylene terephthalates (PET), PET copolyester (PETG), and polyolefins, as well as combinations thereof.

6. A method according to claim 5, wherein said plastic material is polyethylene.

7. A method according to claim 5, wherein said plastic material is polypropylene.

8. A method according to claim 1, wherein said secondary package is made of a material selected from the group consisting of homopolymers or copolymers of chiorotrifluoroethylene (CTFE), polyvinylidene chloride (PVDC), polyethylene vinyl alcohol, polyolefins, polyvinyl chloride, polyethylene terephthalates (PET), PET copolyester (PETG), and combinations thereof.

9. A method according to claim 8, wherein said secondary package is made of a material comprising polyvinyl chloride in combination with homopolymers or copolymers of CTFE or PVDC.

10. A method according to claim 8, wherein said secondary package is made of a material comprising polyolefins in combination with polyethylene vinyl alcohol or PVDC.

11. A method according to claim 8, wherein said secondary package is a sealed blister package.

12. A medicinal product comprising an aqueous composition comprising timolol packaged in a container, which container is made of a pharmaceutically acceptable, moisture-permeable material, characterized in that said container is enclosed in a secondary package, which is made of a pharmaceutically acceptable material having a low permeability to moisture.

13. A medicinal product according to claim 12, wherein said aqueous composition is an ophthalmic composition.

14. A medicinal product according to claim 12, wherein said aqueous composition further comprises a prostaglandin, or an analogue or derivative thereof.

15. A medicinal product according to claim 12, wherein said aqueous composition further comprises latanoprost.

16. A medicinal product according to claim 12, wherein said container at least partially is made of a plastic material selected from the group consisting of polymers and copolymers of polyvinyl chloride, polyethylene terephthalates (PET), PET copolyester (PETG), polyolefins, and combinations thereof.

17. A medicinal product according to claim 16, wherein said plastic material is polyethylene.

18. A medicinal product according to claim 16, wherein said plastic material is polypropylene.

19. A medicinal product according to claim 12, wherein said secondary package is made of a material selected from the group consisting of homopolymers or copolymers of chlorotrifluoroethylene (CTFE), polyvinylidene chloride (PVDC), polyethylene vinyl alcohol, polyolefins, polyvinyl chloride, polyethylene terephthalates (PET), PET copolyester (PETG), and combinations thereof.

20. A medicinal product according to claim 19, wherein said secondary package is made of a material comprising polyvinyl chloride in combination with homopolymers or copolymers of CTFE or PVDC.

21. A medicinal product according to claim 19, wherein said secondary package is made of a material comprising polyolefins in combination with polyethylene vinyl alcohol or PVDC.

22. A medicinal product according to claim 12, wherein said secondary package is a sealed blister package.
